(19) United States
(12) Patent Application Publication
(10) Pub. No.: US 2008/0292612 A1
(43) Pub. Date: Nov. 27, 2008

(54) COMPOSITION CONTAINING SEVERAL BOTULIC TOXINS

(75) Inventor: Laure BERRUET, Chaville (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques (S.C.R.A.S.), Paris (FR)

(21) Appl. No.: 12/180,725

(22) Filed: Jul. 28, 2008

Related U.S. Application Data
(63) Continuation of application No. PCT/FR2007/000134, filed on Jan. 24, 2007.

(30) Foreign Application Priority Data
Jan. 27, 2006 (FR) 06.00732

(51) Int. Cl. A61K 38/48; A61P 21/00; A61Q 19/08

(52) U.S. Cl. 424/94.63

(57) ABSTRACT

The invention concerns a composition including at least one botulinum neurotoxin type A1, and one botulinum neurotoxin type A the amino acid sequence of which as at least 5% difference with the amino acid sequence of botulinum neurotoxin type A1.

This application is a continuation of International Application No. PCT/FR2007/000134, filed on Jan. 24, 2007, the disclosure of which is incorporated by reference.

The present invention involves compositions including several type A botulinum toxins and methods of therapeutic and cosmetic treatment utilizing such composition.

### BACKGROUND OF THE INVENTION

Botulinum toxin, in particular botulinum toxin type A1 (Dysport® marketed by Ipsen or Botox® marketed by Allergan), has been used for treating various diseases/disorders in humans since the 1980s. Botulinum toxin is known to treat numerous disorders including, among others, muscle disorders (for example blepharospasm, spasticity in adults or children or also torticollis), migraine, pain of muscular origin, neuropathic pain, diabetes, hyperhydrosis (or excessive perspiration), hypersalivation and even wrinkles.

The uses of botulinum toxins known at present relate to their standard intra-muscular administration, as described in the treatments mentioned. Injection into the muscles causes their temporary paralysis, i.e. blocks the muscle contractions over a certain period of time.

The botulinum toxin compositions used at present have a period of action which is limited in time.

### BRIEF SUMMARY OF THE INVENTION

In order to respond to industry requirements it has become necessary to find a means for increasing the period of action of the botulinum toxin without altering its biological activity.

Thus, the problem that the invention proposes to solve is to provide a novel botulinum toxin composition having a prolonged period of action.

Unexpectedly, the inventor demonstrates that it is possible to combine several botulinum toxins.

To this end the present invention proposes a composition comprising at least:
one botulinum neurotoxin type A1,
and one botulinum neurotoxin type A the amino acid sequence of which exhibits at least 5% difference from the amino acid sequence of the botulinum neurotoxin type A1.

The invention offers determining advantages, in particular the present composition results in a duration of muscle paralysis which is improved when said composition is injected into a smooth or skeletal muscle. The duration of muscle paralysis is expected to reach up to 10 months in certain cases, and more generally varies between 2 and 8 months.

The invention offers another advantage in that the compositions according to the invention can be used at much lower doses compared with the doses of botulinum toxins which are currently commercially available. In other words, in order to obtain the same relaxation of the muscles, a smaller quantity of botulinum toxin is injected. For the same therapeutic indications, a reduction of between 10 to 70%, preferably between 25 to 50%, in the administration doses is observed (comparison between units of toxins injected in order to obtain the same biological effect).

Another advantage of the compositions according to the invention is that they cause few side-effects, and in particular far fewer side-effects than the botulinum toxin compositions known at present. In particular the possibility of using low doses according to the invention advantageously makes it possible to reduce the side-effects. Among the side-effects which are avoided, there can be mentioned those linked to the immunogenicity of the protein itself, as well as dysphagia, ptosis or general muscle weakness, this list not being exhaustive.

Moreover, the compositions according to the invention have as another advantage an improved speed of action when said composition is injected into a smooth or skeletal muscle. The biological or clinical effect can appear 12 hours after the injection, and more generally within a period of between 14 and 24 hours after the injection.

Advantageously, the compositions according to the invention procure a very homogeneous muscle relaxant effect.

Finally, the invention has the advantage of being able to be utilized in all industries, in particular the pharmaceutical, veterinary and cosmetic industries, as well as in the fields of day-to-day hygiene or body hygiene.

Other advantages and characteristics of the invention will become clearly apparent on reading the following description and examples which are given purely by way of illustration and are non-limitative.

### DETAILED DESCRIPTION OF THE PREFERRED EMBODIMENTS

A subject of the present invention is a composition comprising at least:
one botulinum neurotoxin type A1,
and one botulinum neurotoxin type A, the amino acid sequence of which exhibits at least 5% difference from the amino acid sequence of the botulinum neurotoxin type A1.

The expression “botulinum neurotoxin” means a botulinum toxin which is either a free protein (i.e. free from any protein complexing it), or a protein complex, said protein complex comprising, for example, hemagglutinin (HA protein) combined with the botulinum toxin, or a protein fragment.

The expression “botulinum toxin” means a molecule possessing the biological activity of the botulinum toxin, which can be either a protein, or a polypeptide, or a peptide, or a fusion protein, or a truncated protein, or a chimeric protein, or a mutated protein or a recombinant protein.

The expression “biological activity of the toxin” as used in connection with the present invention means either a muscle paralysis or an inhibition of exocytosis, in particular exocytosis of acetylcholine or another neurotransmitter.

The expression “protein, polypeptide or peptide” as used in connection with the present invention means a polymer of amino acids, natural or non-natural, levo-rotatory or not levo-rotatory, dextro-rotatory or not dextro-rotatory.

The expression “chimeric protein” as used in connection with the present invention means a protein obtained after combination of different types of molecules, for example after combination of lipids, glycolipids, peptides, polypeptides, proteins, glycoproteins, carbohydrates, polysaccharides, nucleic acids, polyethylene glycol, etc.

Botulinum neurotoxin, pure or virtually pure, can be obtained from a protein complex comprising botulinum toxin for example according to the method described in Current topics in Microbiology and Immunology (1995), 195, p. 151-154. A botulinum neurotoxin, pure or virtually pure, can be obtained for example, by purification of a fermentation medium or culture broth containing a strain of Clostridium botulinum, and enriched for example with meat or protein-rich food.

The first essential constituent of the composition is constituted by a botulinum neurotoxin type A1.

The botulinum neurotoxin type A1 corresponds in fact to the standard botulinum toxin which is commonly called botulinum toxin type A, without distinguishing the sub-type. The botulinum neurotoxin type A1 is marketed under the name of DYSPORT® or BOTOX®.

According to the invention, the botulinum neurotoxin type A1 can correspond either to a complex of botulinum toxin A1 and hemagglutinin, or to the botulinum toxin type A1 which is free of all complexing proteins.

The second essential constituent of the composition is constituted by a botulinum neurotoxin type A, the amino acid sequence of which exhibits at least 5% difference from the amino acid sequence of the botulinum neurotoxin type A1. To facilitate reading of the remainder of the disclosure, this neurotoxin is referred to as botulinum neurotoxin type Ax.

The expression “a botulinum neurotoxin type A the amino acid sequence of which exhibits at least λ% difference from the amino acid sequence of the botulinum neurotoxin type A1” means that these amino acid sequences (heavy chain and light chain) correspond to the sequences of the botulinum toxin which is free of all complexing proteins. For example, 10% difference means that 10 amino acids out of 100 amino acids are different. It is understood that different can mean missing. Thus, in the example, 10% difference can mean 10 amino acids missing out of 100 amino acids.

More particularly the composition according to the invention comprises at least one botulinum neurotoxin type A the amino acid sequence of which exhibits at least 8% difference from the amino acid sequence of the botulinum neurotoxin type A1.

Preferably the composition according to the invention comprises at least one botulinum neurotoxin type A the amino acid sequence of which exhibits at least 10% difference from the amino acid sequence of the botulinum neurotoxin type A1.

Still more preferentially the composition according to the invention comprises at least one botulinum neurotoxin type A the amino acid sequence of which exhibits at least 12% difference from the amino acid sequence of the botulinum neurotoxin type A1.

Among the botulinum neurotoxins type Ax, there can be mentioned the botulinum toxin type A2.

Preferably, the composition according to the invention comprises at least:
one botulinum neurotoxin type A1, and
one botulinum neurotoxin type A2.

Botulinum toxin type A2 was first isolated starting with cases of children suffering from botulism around 1990 (Sakaguchi et al., Int. J. Food Microbiol. (1990), 11, 231-242). This toxin is immunologically and biochemically different from botulinum toxin type A1.

Botulinum toxin type A2 can be isolated from the following strains: Kyoto-F, Chiba-H, Y-8036, 7103-H, 7105-H, KZ1828, NCTC2012 or NCTC9837 (Cordoba et al., System. Appl. Microbiol. (1995), 18, 13-22; Franciosa et al., abstract presented at 40th Interagency Botulism Research Coordinating Committee (IBRCC) Meeting, November 2003).

Preferably the composition according to the invention comprises botulinum toxin type A2 isolated from the strain Clostridium botulinum referenced and accessible under the number NCTC9837, at the National Collection of Type Cultures—Central Public Health Laboratory—London—UK. The strain NCTC9837 is sometimes called strain Mauritius 1955.

Still more preferentially, the composition according to the invention comprises at least:
one botulinum neurotoxin type A1, and
one botulinum neurotoxin type A2 isolated from the strain Clostridium botulinum NCTC9837.

Botulinum toxin type A2 differs from toxin A1 by, inter alia, its amino acid sequence, its molecular weight, its immunological and genetic characteristics (Kubota et al., Biochem. Biophys. Res. Commun. (1996), 224 (3), 843-848).

The biochemical differences between botulinum toxins type A2 and type A1 are inter alia that botulinum toxin type A1 contains the NTNH protein (non-toxic and non-hemagglutinin protein) and at least 3 hemagglutinins (HA17, HA34 and HA70), while botulinum toxin type A2 can contain the NTNH protein, without hemagglutinin (Sakaguchi et al., Int. J. Food Microbiol. (1990), 11, 231-242).

Botulinum toxin type A2 possesses a heavy chain of approximately 101 kDa while botulinum toxin type A1 possesses a heavy chain of approximately 93 kDa (Kozaki et al., Microbiol. Immunol. (1995), 39(10), 767-74).

The amino acid sequence of botulinum toxins type A2 and A1 is significantly different, more particularly at the level of the heavy chain. For example in the case of botulinum toxin type A2 originating from the strain Kyoto-F, 109 amino acids out of the 847 of the amino acids of the heavy chain are different between the 2 toxins (13% difference) (Cordoba et al., System. Appl. Microbiol. (1995), 18, 13-22).

Generally, the heavy chains of isolated botulinum toxins type A1 differ from each other by less than 2% difference. The heavy chain of the botulinum toxin is involved in the main biological activities of the molecule, including binding to the receptor on the target cells and intracellular transport (Zhang et al., Gene (2003), 315, 21-32).

Immunological differences exist between botulinum toxins type A2 and type A1. In fact antibodies directed against botulinum toxin type A1 do not recognize botulinum toxin type A2, and vice-versa. (Sakaguchi et al., Int. J. Food Microbiol. (1990), 11, 231-242; Kozaki et al., Microbiol. Immunol. (1995), 39(10), 767-74).

The present composition according to the invention can moreover comprise at least one analgesic agent.

Among the suitable analgesic agents according to the present invention there can be mentioned an analgesic agent chosen from the inhibitors of the sodium channels.

The present composition according to the invention can also comprise a mixture of analgesic agents mentioned below.

The expression “inhibitors of the sodium channels” includes for example the following compounds (optionally in the form of pharmaceutically acceptable salts):
lidocaine;
tetracaine;
bupivacaine;
procaine;
mepivacaine; or
dibucaine;

The present composition according to the invention can also comprise at least one polysaccharide or a mixture of several polysaccharides.

The term “polysaccharide” as used in connection with the present invention means a polymer comprising at least 2 monomers, the monomers being saccharides. This definition includes the disaccharides.

Within the framework of the invention, the polysaccharides can be ionic and/or non-ionic.

Preferably, the composition comprises at least one polysaccharide comprising mostly glucose units, the term “mostly” meaning that, in numerical terms, the majority of the monomer units are glucose.

As examples of suitable polysaccharides according to the invention, there can be mentioned starch, starch derivatives, hydroxyethyl starch in particular 2-hydroxy-ethyl starch.

The suitable polysaccharides according to the present invention can be substituted, in particular substituted by alkyl, alkoxy radicals, or also by alkyl radicals themselves substituted by alcohol functions.

According to a variant of the invention, the quantity of polysaccharide comprised in the composition according to the invention is at least 1 μg of polysaccharide per 1 unit of botulinum toxin. According to the choice of the polysaccharide, it is possible to use at least 0.5 μg of polysaccharide per 1 unit of botulinum toxin.

The present composition according to the invention can moreover comprise at least one surfactant or a mixture of several surfactants.

By “surfactant” is meant within the meaning of the invention an emulsifying agent or a solubilizing agent.

Within the framework of the invention the surfactants utilized can be chosen from the cationic, anionic or non-ionic surfactants.

Preferably the composition according to the invention comprises at least one surfactant chosen from the non-ionic surfactants of the group of polysorbates.

Among the group of polysorbates, there can be mentioned polysorbate 20, polysorbate 21, polysorbate 40, polysorbate 60, polysorbate 61, polysorbate 65, polysorbate 80, polysorbate 81, polysorbate 85, polysorbate 120, polysorbate 80 acetate.

The preferred surfactant according to a variant of the composition according to the invention is polysorbate 80.

The present composition according to the invention can moreover comprise at least one vasoconstrictor or a mixture of several vasoconstrictors.

Within the framework of the invention the vasoconstrictors utilized can be chosen for example from the vasoconstrictors of the catecholamine or non-catecholamine type.

Among the suitable vasoconstrictors according to the present invention, there can be mentioned as examples epinephrine, norepinephrine, levonordefrin, amphetamines, ephedrine, phenylephrine, endothelin.

The composition according to the invention can be in the form of a solid, for example powders, lyophilizates, granules, tablets or liposomes. The composition according to the invention in the solid form can be preserved for example at temperatures of less than 4° C., or less than 0° C. without its biological activity being altered.

The composition according to the invention can be presented in the form of an aqueous dispersion, of particles of botulinum neurotoxin in a gelified system.

The composition according to the invention can also be presented in liquid form, for example, solutions, emulsions or suspensions.

The administration of the composition according to the invention is preferably carried out by injection such as for example by intramuscular or sub-cutaneous injection.

In the case of injections, the composition according to the invention can be combined with an agent facilitating the injection also called an injection vehicle or injection vector.

According to an embodiment of the compositions according to the invention, the ratio of the fractions of units of botulinum neurotoxin type A1 to botulinum neurotoxin type Ax [units A1/units Ax] can be comprised between 1/99 and 99/1, advantageously between 5/95 and 95/5, or between 10/90 and 90/10.

According to a variant of the composition according to the invention, the ratio of the fractions of units of botulinum neurotoxin type A1 to botulinum neurotoxin type Ax is 50/50.

According to another variant of the composition according to the invention, the ratio of the fractions of units of botulinum neurotoxin type A1 to botulinum neurotoxin type Ax is 60/40.

According to another variant of the composition according to the invention, the ratio of the fractions of units of botulinum neurotoxin type A1 to botulinum neurotoxin type Ax is 40/60.

A subject of the present invention is also the use of a composition according to the invention, described above, for obtaining a medicament intended to treat muscular disorders, neuromuscular disorders, neurological disorders, orthopaedic disorders, ophthalmological disorders, articular pathologies, endocrine disorders or urological disorders.

A subject of the present invention is also the use of a composition according to the invention, described above, for obtaining a medicament intended to treat torticollis, spasmodic torticollis, local disorders of spasticity of the upper and/or lower limbs, pain, muscle pain, pain due to muscle spasms, myofascial pain, post-operative pain, muscle spasms, hemifacial spasm, blepharospasm, strabismus, facial asymmetry, muscle dystonia, cerebral paralysis, headaches, migraine, fibromyalgia, myalgia, hyperhidrosis, bromhidrosis, coxarthrosis, arthrosis of the hip, epicondylitis of the elbow, arthritis, rheumatoid arthritis, dyskinesias, achalasia, Oddi's sphincter dysfunctions, pancreatitis, gout, anal fissures, constipation, anismus, spasms of the pyloric valve, spastic bladder, spasms of the bladder, urinary incontinence, urine retention, prostatic hyperplasia, endometriosis, psoriasis, rhinitis, allergic rhinitis, obesity, hyperlacrimation, bone fractures, tendon lacerations or rotator muscle cap pathology of the shoulder.

A subject of the present invention is also the use of a composition according to the invention, described above, for obtaining a cosmetic product.

A subject of the present invention is also the use of a composition according to the invention, described above, for treating facial frown lines, facial wrinkles, wrinkles of the skin, wrinkles of the contour of the eye, glabellar frown lines, baldness, acne, excessive perspiration or hair loss.

A subject of the present invention is also, as a medicament, the composition according to the invention described above.

A subject of the present invention is also a pharmaceutical composition comprising the composition according to the invention described above.

The dose of the composition according to the present invention, to be provided for the treatment of the diseases or disorders mentioned above, varies according to the administration method, the age and body weight of the subject to be treated as well as the state of the latter, and will be finally decided by the attending doctor or vet. Such a quantity determined by the attending doctor or vet is here called the “therapeutically effective quantity”.

The therapeutically effective quantity which should be injected also varies according to the number of muscles to be treated, as well as according to the mass of these muscles.

Preferably, the injected doses of composition according to the invention are comprised between 5 and 2000 units of botulinum toxin, more preferentially 10 to 1000 units of botulinum toxin, still more preferentially 25 to 500 units of botulinum toxin.

The following examples illustrate the invention without limiting its scope.

### EXAMPLES

The quantification of the botulinum neurotoxins used according to the invention is carried out by measuring a lethal dose LD₅₀. By “LD₅₀” is meant within the meaning of the present invention the lethal dose or also semi-lethal dose of a given substance. It is the dose (or quantity) which leads to the death of 50% of the animals tested in a group. A unit of toxin (U) corresponds to the LD₅₀ in mice by intra-peritoneal route.

Compositions Used in the Examples:

The essential constituents of the compositions used in the examples are described below. The other constituents present have not been specified.

Composition A:—250 units of botulinum neurotoxin type A1 (Dysport®)
250 units of botulinum neurotoxin type A2 isolated from the strain Clostridium botulinum NCTC9837

Composition B:—500 units of botulinum neurotoxin type A1 (Dysport®)

### Example 1

### Blepharospasm (Dose 120 Units):

A fifty-year-old patient suffering from blepharospasm, involuntary closing of the eyes resulting from the contraction of the muscles situated around the eyes, is given several intramuscular injections of composition A according to the invention, the total injected dose of composition A being 120 units of neurotoxin botulinum for a total volume of 1 ml.

The injection protocol includes the following steps: a sterile needle is introduced into a vial containing composition A in order to introduce into it 2.5 ml of an injectable 0.9% sodium chloride solution. Thus a clear solution containing 200 units/ml of botulinum neurotoxin is obtained.

A patient suffering from bilateral blepharospasm is given, by injection, 120 units of botulinum neurotoxin (composition A) per eye (i.e. 0.6 ml, for the dilution of 1 vial containing 500 units of botulinum neurotoxin (composition A) in 2.5 ml). The injections are administered, after cleansing the skin around the eyes, by sub-cutaneous route with a 1 ml syringe (23 or 25 gauge needle).

A dose of 0.1 ml (20 units) of composition A is injected into the internal part and a dose of 0.2 ml (40 units) of composition A into the external part of the junction between the preseptal and orbital zones of the upper orbicularis oculi muscles of each eye. In the same way a dose of 0.1 ml (20 units) of composition A is injected into the internal part and a dose of 0.2 ml (40 units) of composition A into the external part of the junction between the preseptal and orbital zones of the lower orbicularis oculi muscles of each eye. During the injection into the upper eyelid, the needle must preferably be oriented such that the centre of the eyelid, the part where the levator palpebrae superioris muscle inserts, is avoided. During subsequent administrations, the total dose per eye can be reduced to 80 units (0.4 ml), i.e. 20 units per injection site (i.e. 0.1 ml).

In the case of this treatment, muscle paralysis commences approximately 12 hours after the start of the treatment. The paralysis lasts approximately 20 to 21 weeks with no partial or complete ptosis being observed.

In the case of another patient of the same age and presenting with similar symptoms, with the same protocol but using composition B with the same dosage, the muscle paralysis commences approximately 24 hours after the start of the treatment, and lasts approximately 12 to 13 weeks.

### Example 2

### Blepharospasm (Dose 60 Units):

A fifty-year-old patient suffering from Blepharospasm, involuntary closing of the eyes resulting from the contraction of the muscles situated around the eyes, is given several intramuscular injections of composition A according to the invention, the total injected dose of composition A being 60 units of botulinum neurotoxin for a total volume of 1 ml.

The injection protocol includes the following steps: a sterile needle is introduced into a vial containing composition A in order to introduce into it 2.5 ml of an injectable solution of 0.9% sodium chloride. A clear solution containing 200 units/ml of botulinum neurotoxin is thus obtained.

A patient suffering from bilateral blepharospasm is injected with 60 units of botulinum neurotoxin (composition A) per eye (i.e. 0.3 ml, for the dilution of 1 vial containing 500 units of botulinum neurotoxin (composition A) in 2.5 ml). The injections are administered, after cleansing of the skin around the eyes, by sub-cutaneous route.

A dose of 0.05 ml (10 units) of composition A is injected into the internal part and a dose of 0.1 ml (20 units) of composition A into the external part of the junction between the preseptal and orbital zones of the upper orbicularis oculi muscles of each eye. In the same way, a dose of 0.05 ml (10 units) of composition A is injected into the internal part and a dose of 0.1 ml (20 units) of composition A into the external part of the junction between the presentable and orbital zones of the lower orbicularis oculi muscles of each eye. During the injection into the upper eyelid, the needle must preferably be oriented such that the centre of the eyelid, the part where the levator palpebrae superioris muscle inserts, is avoided. During subsequent administrations, the total dose per eye can be reduced to 40 units (0.2 ml), i.e. 10 units per injection site (i.e. 0.05 ml).

In the case of this patient, the muscle paralysis commences approximately 24 hours after the start of the treatment, and the paralysis lasts approximately 12 to 13 weeks.

The results are therefore similar to those obtained with composition B, for an administration dose reduced by 50% by number of toxin units.

### Example 3

### Spasmodic Torticolis

A 35-year-old female patient suffering from spasmodic torticollis is given several intramuscular injections of composition A according to the invention, the total injected dose of composition A being 500 units of botulinum neurotoxins for a total volume of 1 ml.

The injection protocol includes the following steps: a sterile needle is introduced into a vial containing composition A in order to introduce into it 1 ml of an injectable 0.9% sodium chloride solution. Thus a clear solution containing 500 units/ml of botulinum neurotoxin is obtained.

A female patient suffering from spasmodic torticollis is given, by injection, 500 units of botulinum neurotoxin (composition A) i.e. 1 ml, for the dilution of 1 vial of 500 units in 1 ml. The total dose was distributed between the 2 or 3 most-affected cervical muscles (sternocleidomastoidian, splenius, trapezius or angular).

In this patient the muscle paralysis would be expected to commence approximately 18 hours after the start of the treatment. The paralysis would be expected to last approximately 20 to 21 weeks. No dysphagia was noted.

In another female patient of the same age and presenting with similar symptoms, with the same protocol but using composition B with the same dosage, the muscle paralysis commences approximately 48 to 60 hours after the start of the treatment, and the paralysis lasts approximately 12 to 13 weeks.

1. A composition comprising at least:
one botulinum neurotoxin type A1, and
one botulinum neurotoxin type A, the amino acid sequence of which exhibits at least 5% difference from the amino acid sequence of the botulinum neurotoxin type A1.

2. The composition according to claim 1, wherein the composition comprises at least one botulinum neurotoxin type A, the amino acid sequence of which exhibits at least 10% difference from the amino acid sequence of the botulinum neurotoxin type A1.

3. The composition according to claim 1, wherein the composition comprises at least
one botulinum neurotoxin type A1, and
one botulinum neurotoxin type A2.

4. The composition according to claim 1, wherein the composition comprises at least
one botulinum neurotoxin type A1, and
one botulinum neurotoxin type A2 isolated from the strain Clostridium botulinum NCTC9837.

5. The composition according to claim 1, wherein the composition comprises at least one analgesic agent.

6. The composition according to claim 1, wherein the composition comprises at least one polysaccharide.

7. The composition according to claim 6, wherein the polysaccharide is 2-hydroxyethyl starch.

8. The composition according to claim 1, wherein the composition comprises at least one cationic, anionic or non-ionic surfactant.

9. The composition according to claim 1, wherein the composition comprises at least one polysorbate non-ionic surfactant.

10. The composition according to claim 1, wherein the composition comprises at least one surfactant.

11. The composition according to claim 1, wherein the composition comprises at least one vasoconstrictor.

12. A medicament composition comprising the composition according to claim 1.

13. A method of treatment of muscular disorders, neuromuscular disorders, neurological disorders, orthopaedic disorders, ophthalmological disorders, articular pathologies, endocrine disorders or urological disorders comprising administering the composition of claim 1 in a therapeutically effective quantity for treating such disorder to a patient suffering from a disorder comprising.

14. A method of treatment of torticollis, spasmodic torticollis, local disorders of spasticity of the upper and/or lower limbs, pain, muscle pain, pain due to muscle spasms, myofascial pain, post-operative pain, muscle spasms, hemifacial spasm, blepharospasm, strabismus, facial asymmetry, muscle dystonia, cerebral paralysis, headaches, migraine, fibromyalgia, myalgia, hyperhidrosis, bromhidrosis, coxarthrosis, arthrosis of the hip, epicondylitis of the elbow, arthritis, rheumatoid arthritis, dyskinesias, achalasia, Oddi's sphincter dysfunctions, pancreatitis, gout, anal fissures, constipation, anismus, spasms of the pyloric valve, spastic bladder, spasms of the bladder, urinary incontinence, urine retention, prostatic hyperplasia, endometriosis, psoriasis, rhinitis, allergic rhinitis, obesity, hyperlacrimation, bone fractures, tendon lacerations or rotator muscle cap pathology of the shoulder comprising administering the composition of claim 1 in a therapeutically effective quantity for treating such disorder to a patient suffering from a disorder comprising.

15. A method of cosmetic treatment comprising administering the composition of claim 1 to a patent desiring such treatment.

16. A method of treating facial frown lines, facial wrinkles, wrinkles of the skin, wrinkles of the contour of the eye, glabellar frown lines, baldness, acne, excessive perspiration or hair loss comprising administering the composition of claim 1 in an amount effective to treat such condition.

17. A method of treating blepharospasm comprising administering the composition of claim 1 in a therapeutically effective quantity for treatment of blepharospasm.

18. A method of treating spasmodic torticollis comprising administering the composition of claim 1 in a therapeutically effective quantity for treatment of spasmodic torticollis.

19. A method of cosmetic treatment comprising administering the composition of claim 4 to a patent desiring such treatment.

20. A method of treating facial frown lines, facial wrinkles, wrinkles of the skin, wrinkles of the contour of the eye, glabellar frown lines, baldness, acne, excessive perspiration or hair loss comprising administering the composition of claim 4 to a patent in an amount effective to treat such condition.

21. A method of treating blepharospasm comprising administering to a patient the composition of claim 4 in a therapeutically effective quantity for treatment of blepharospasm.

22. A method of treating spasmodic torticollis comprising administering to a patient the composition of claim 4 in a therapeutically effective quantity for treatment of spasmodic torticollis.

23. The composition of claim 10, wherein said surfactant is polysorbate 80.

24. The medicament of claim 12, wherein the composition is an aqueous solution.
